(19) United States
(12) Patent Application Publication
(10) Pub. No.: US 2014/0074059 A1
(43) Pub. Date: Mar. 13, 2014

(54) METHOD AND SYSTEM TO INDICATE GLYCEMIC IMPACTS OF INSULIN INFUSION PUMP COMMANDS

(71) Applicants: Frances Wilson HOWELL, Chester Springs, PA (US); Janice MACLEOD, Chester Springs, PA (US); David RODBARD, Potomac, MD (US)

(72) Inventors: Frances Wilson HOWELL, Chester Springs, PA (US); Janice MACLEOD, Chester Springs, PA (US); David RODBARD, Potomac, MD (US)

(73) Assignee: LifeScan, Inc., Milpitas, CA (US)

(21) Appl. No.: 13/612,506

(22) Filed: Sep. 12, 2012

(51) Int. Cl. A61M 5/168

(52) U.S. Cl. 604/503; 604/66

(57) ABSTRACT

Various methods and system to provide insight into how certain commands to a patient's infusion pump impact glycemic control of the subject. These patterns help to identify very specific areas of glycemic excursions, enable patients and HCPs to more easily identify patterns of hypoglycemia and hyperglycemia in order to take steps to improve glycemic control of the person with diabetes.

### BACKGROUND

Glucose monitoring is a fact of everyday life for many people with diabetes. The accuracy of such monitoring can significantly affect the health and ultimately the quality of life for people with diabetes. A person with diabetes may measure blood glucose levels several times a day as a part of the diabetes self management process. Failure to maintain target glycemic control can result in serious diabetes-related complications, including cardiovascular disease, kidney disease, nerve damage and blindness. There are a number of electronic devices currently available which enable an individual to check the glucose level in a small sample of blood. One such glucose meter is the OneTouch® Verio® glucose meter, a product which is manufactured by LifeScan.

In addition to glucose monitoring, people with diabetes often have to administer drug therapy such as insulin. People with diabetes self-administer insulin to manage their blood glucose concentration. There are a number of mechanical devices currently available which enable an individual to dose a predetermined quantity of insulin such as a hypodermic syringe, an insulin pen and an insulin pump. One such insulin pump is the OneTouch® Ping, a product which is manufactured by Animas Corporation. Another is the Animas® Vibe, also manufactured by Animas Corporation.

People with diabetes should maintain tight control over their lifestyle, so that they are not adversely affected by certain lifestyle choices such as irregular food consumption or exercise. In addition, a health care professional (HCP) dealing with a person with diabetes may require detailed information on the individual's lifestyle to provide effective treatment or modification of treatment for managing diabetes. Currently, one of the ways of monitoring the lifestyle of an individual with diabetes has been for the individual to keep a paper logbook of their lifestyle. Another way is for an individual to simply rely on remembering facts about their lifestyle and then relay these details to their HCP at each visit.

The aforementioned methods of recording lifestyle information are inherently difficult, time consuming and possibly inaccurate. Paper logbooks are not necessarily always carried by an individual and may not be accurately completed when required. Such paper logbooks are small and it is therefore difficult to enter the detailed information required of lifestyle events. Furthermore, an individual may often forget key facts about their lifestyle when questioned by a HCP who has to manually review and interpret information from a hand-written notebook. There is no analysis provided by the paper logbook to distill or separate the component information. Also, there are no graphical reductions or summary of the information. Entry of data into a secondary data storage system, such as a database or other electronic system requires a laborious transcription of information, including lifestyle data, into this secondary data storage. Difficulty of data recordation encourages retrospective entry of pertinent information that results in inaccurate and incomplete records.

### SUMMARY OF THE DISCLOSURE

In one embodiment, a system for management of diabetes of a subject is provided. The system includes at least one glucose monitor for measurements of the glucose levels of the subject, an insulin infusion pump configured for communication with the at least one glucose monitor and delivery of insulin to the subject; and a controller in communication with at least the insulin infusion pump and the at least one glucose monitor. The controller is configured or programmed to receive or transmit data regarding glucose levels and dosing of insulin from the at least one glucose monitor and pump for analysis by the controller so that at least one of a plurality of patterns in glucose due to at least one of a plurality of pump commands is determined via the controller by: determination of whether there is at least one glucose measurement made within a predetermined time interval after occurrence of one of a plurality of pump commands; flag the at least one glucose measurement as a flagged high measurement whenever the at least one glucose measurement is equal to or greater than a high threshold; flag the at least one glucose measurement as a flagged low measurement whenever the at least one glucose measurement is equal to or less than a low threshold; calculate a percentage of flagged high glucose measurements from total glucose measurements made during the predetermined time interval over a plurality of days; calculate a percentage of flagged low glucose measurement from total glucose measurements made during the predetermined time interval over a plurality of days; annunciate at least a first message that a high glucose pattern has been detected in relation to the one of a plurality of pump commands whenever the percentage of flagged high glucose measurements is equal to or greater than a first percentage or a second message that a low glucose pattern has been detected in relation to the one of a plurality of pump commands whenever the percentage of flagged low glucose measurements is equal to or greater than a second percentage.

In another embodiment, a method for managing diabetes of a subject with at least a glucose monitor is provided. The method can be achieved by: conducting, with the glucose monitor, a plurality of glucose measurements of the subject; storing the plurality of glucose measurements in a memory; determining whether there is at least one glucose measurement made within a predetermined time interval after occurrence of one of a plurality of pump commands; flagging the at least one glucose measurement as a flagged high measurement whenever the at least one glucose measurement is equal to or greater than a high threshold; flagging the at least one glucose measurement as a flagged low measurement whenever the at least one glucose measurement is equal to or less than a low threshold; calculating a percentage of flagged high glucose measurements from total glucose measurements made during the predetermined time interval over a plurality of days; calculating a percentage of flagged low glucose measurements from total glucose measurements made during the predetermined time interval over a plurality of days; annunciating at least a first message that a high glucose pattern has been detected in relation to the one of a plurality of pump commands whenever the percentage of flagged high glucose measurements is equal to or greater than a first percentage or a second message that a low glucose pattern has been detected in relation to the one of a plurality of pump commands whenever the percentage of flagged low glucose measurements is equal to or greater than a second percentage.

In yet a further aspect, a method for managing diabetes of a subject with at least a glucose monitor and infusion pump. The method can be achieved by: conducting, with the glucose monitor, a plurality of glucose measurements of the subject; storing the plurality of glucose measurements in a memory; determining whether there is at least one glucose measurement made during a predetermined time period after occurrence of a pump suspend command and a second predetermined time period after occurrence of a pump resume command; flagging the at least one glucose measurement as a flagged high measurement whenever the at least one glucose measurement is equal to or greater than a high threshold; calculating a percentage of flagged high glucose measurements from total glucose measurements made during the first and second predetermined time periods over a plurality of days; annunciating a first message that a high glucose pattern has been detected in relation to the pump suspend command whenever the percentage of flagged high glucose measurements is equal to or greater than a first percentage.

In another aspect, a method for managing diabetes of a subject with at least a glucose monitor and infusion pump is provided. The method can be achieved by: conducting, with the glucose monitor, a plurality of glucose measurements of the subject; storing the plurality of glucose measurements in a memory; determining whether there is at least one glucose measurement made within a predetermined time interval after occurrence of a pump prime command; flagging the at least one glucose measurement as a flagged low measurement whenever the at least one glucose measurement is equal to or less than a low threshold; calculating a percentage of flagged low glucose measurements from total glucose measurements made during the predetermined time interval over a plurality of days; and annunciating a second message that a low glucose pattern has been detected in relation to the pump prime command whenever the percentage of flagged low glucose measurements is equal to or greater than a second percentage.

In each of the aspects or embodiments described above, the following features may be combined in various permutations. For example, the plurality of pump commands may include a command for: a bolus based on a carbohydrate calculator, a bolus based on measured glucose values, an override of a programmed bolus, a programmed bolus, or a temporary basal rate; the first percentage may include about 50% and the second percentage may include about 5%; the calculating of the percentage of flagged high glucose measurements may include dividing the number of flagged high glucose measurements by a total number of glucose measurements made during the predetermined time interval for a plurality of days multiplied by 100 and the calculating of the percentage of flagged low glucose measurements may include dividing the number of flagged low glucose measurements by a total number of glucose measurements made during the predetermined time interval for a plurality of days multiplied by 100; the plurality of pump commands may include a command for: a bolus based on a carbohydrate calculator, a bolus based on measured glucose values, an override of a programmed bolus, a programmed bolus, or a temporary basal rate; the first percentage may include about 50% and the second percentage may include about 5%; the first and second time period comprise equal time intervals; the first and second time period comprise unequal time intervals; each of the first and second time periods may include about one hour in duration; the calculating of the percentage of flagged high glucose measurements may include dividing the number of flagged high glucose measurements by a total number of glucose measurements made during the predetermined time periods for a plurality of days multiplied by 100; the first percentage may include about 50%; the calculating of the percentage of flagged low glucose measurements may include dividing the number of flagged low glucose measurements by a total number of glucose measurements made during the predetermined time interval for a plurality of days multiplied by 100; the second percentage may include about 5%; the predetermined time interval may include about 2 hours.

These and other embodiments, features and advantages will become apparent to those skilled in the art when taken with reference to the following more detailed description of various exemplary embodiments of the invention in conjunction with the accompanying drawings that are first briefly described.

### BRIEF DESCRIPTION OF THE FIGURES

The accompanying drawings, which are incorporated herein and constitute part of this specification, illustrate presently preferred embodiments of the invention, and, together with the general description given above and the detailed description given below, serve to explain features of the invention (wherein like numerals represent like elements).

FIG. 1 illustrates in schematic form the software engine to determine hypoglycemia or hyperglycemia of a subject based on input data from either or both of at least a glucose monitor and an insulin infusion pump.

FIG. 2 illustrates an exemplary glucose management system that can be used with the software engine of FIG. 1.

FIG. 3 illustrates the logic to detect patterns impacting the glycemic state of users due to certain eZ Carb Bolus pump command(s) in the system of FIG. 2.

FIG. 4 illustrates the logic to detect patterns impacting the glycemic state of users due to certain ezBG-Bolus pump command(s) in the system of FIG. 2.

FIG. 5 illustrates the logic to detect patterns impacting the glycemic state of users due to certain normal bolus pump command(s) in the system of FIG. 2.

FIG. 6 illustrates the logic to detect patterns impacting the glycemic state of users due to certain bolus override pump command(s) in the system of FIG. 2.

FIG. 7 illustrates the logic to detect patterns impacting the glycemic state of users due to certain cannula fill pump command(s) in the system of FIG. 2.

FIG. 8 illustrates the logic to detect patterns impacting the glycemic state of users due to certain suspend pump command(s) in the system of FIG. 2.

FIG. 9 illustrates the logic to detect patterns impacting the glycemic state of users due to certain temporary basal rate pump command(s) in the system of FIG. 2.

FIG. 10 illustrates the logic to detect patterns impacting the glycemic state of users due to certain pump prime command(s) in the system of FIG. 2.

### MODES FOR CARRYING OUT THE INVENTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. In addition, as used herein, the terms “patient,” “host,” “user,” and “subject” refer to any human or animal subject and are not intended to limit the systems or methods to human use, although use of the subject invention in a human patient represents a preferred embodiment.

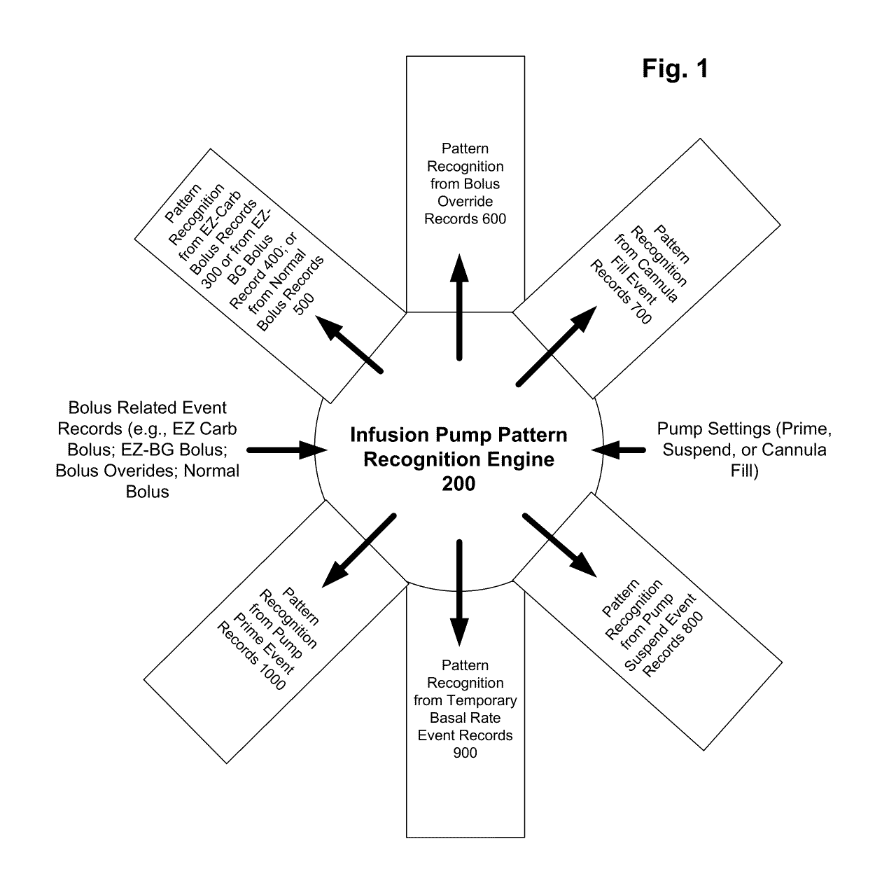

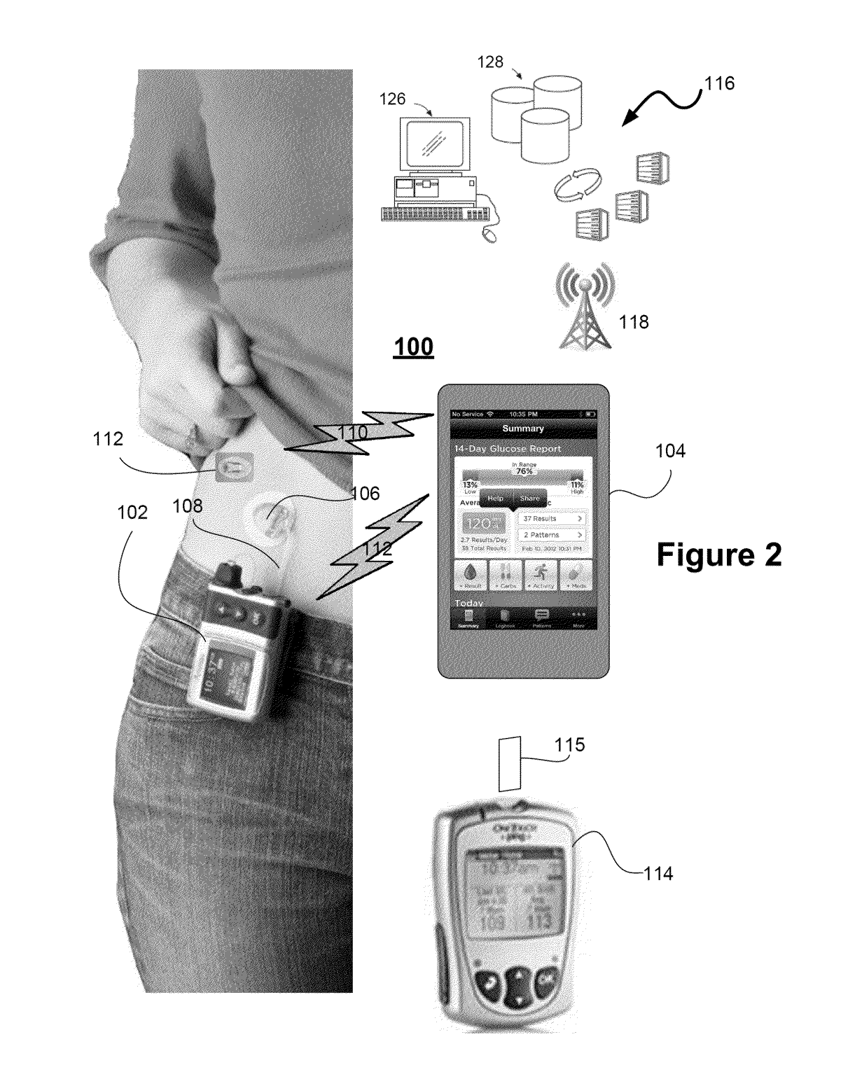

FIG. 1 illustrates a software engine 200 configured for use with microprocessor-enabled components of the FIG. 2. The software engine 200 receives a plurality of inputs to allow the software to recognize physiological impacts (in the form of blood glucose values) from usage of the insulin pump. In particular, the inputs to the engine 200 may include records of pump commands or pump event records such as, for example, bolus dosage based on estimated carbohydrates intake, bolus dosage based on blood glucose readings, bolus overrides, pre-programmed or normal bolus dosage, priming of the pump, suspending infusion of insulin, or to fill the cannula. Of course, one critical input is blood glucose (“BG”) values derived from either a discontinuous glucose monitor (e.g., glucose test meter and strips) or a continuous glucose monitor. The engine 200 is configured to recognize various patterns impacting the glycemic state of the patient or user from the inputs such as, for example, certain pump commands that will be later described.

FIG. 2 illustrates a drug delivery system 100 according to an exemplary embodiment. Drug delivery system 100 includes a drug delivery device 102 and a remote controller 104. Drug delivery device 102 is connected to an infusion set 106 via flexible tubing 108. Drug delivery device 102 is configured to transmit and receive data to and from remote controller 104 by, for example, radio frequency communication 110. Drug delivery device 102 may also function as a stand-alone device with its own built in controller.

In one embodiment, drug delivery device 102 may include a drug infusion device and remote controller 104 may include a hand-held portable controller. In such an embodiment, data transmitted from drug delivery device 102 to remote controller 104 may include information such as, for example, drug delivery data, blood glucose information, basal insulin delivery, bolus insulin delivery, insulin to carbohydrates ratio or insulin sensitivity factor, to name a few. The controller 104 is configured to include a controller that has been programmed to receive continuous analyte readings from a CGM sensor 112. Data transmitted from remote controller 104 to drug delivery device 102 may include analyte test results and a food database to allow the drug delivery device 102 to calculate the amount of drug to be delivered by drug delivery device 102. Alternatively, the remote controller 104 may perform basal dosing or bolus calculation and send the results of such calculations to the drug delivery device. In an alternative embodiment, an episodic blood analyte meter 114 may be used alone or in conjunction with the CGM sensor 112 to provide data to either or both of the controller 104 and drug delivery device 102. Alternatively, the remote controller 104 may be combined with the meter 114 into either (a) an integrated monolithic device; or (b) two separable devices that are dockable with each other to form an integrated device. Each of the devices 102, 104, and 114 has a suitable micro-controller (not shown for brevity) programmed to carry out various functionalities.

Drug delivery device 102 may also be configured for bi-directional wireless communication with a remote health monitoring station 116 through, for example, a wireless communication network 118. Remote controller 104 and remote monitoring station 116 may be configured for bi-directional wired communication through, for example, a telephone land based communication network. Remote monitoring station 116 may be used, for example, to download upgraded software to drug delivery device 102 and to process information from drug delivery device 102. Examples of remote monitoring station 116 may include, but are not limited to, a personal or networked computer 126, server 128 to memory storage, a personal digital assistant, other mobile telephone, a hospital base monitoring station or a dedicated remote clinical monitoring station.

Drug delivery device 102 includes certain components including a central processing unit, memory elements for storing control programs and operation data, a radio frequency module 116 for sending and receiving communication signals (i.e., messages) to/from remote controller 104, a display for providing operational information to the user, a plurality of navigational buttons for the user to input information, a battery for providing power to the system, an alarm (e.g., visual, auditory or tactile) for providing feedback to the user, a vibrator for providing feedback to the user, and a drug delivery mechanism (e.g. a drug pump and drive mechanism) for forcing a drug from a drug reservoir (e.g., a drug cartridge) through a side port connected to an infusion set 106 and into the body of the user. Other suitable infusers can also be utilized such as, for example, a basal and bolus patch pump or even an infusing pen can also be utilized.

Analyte levels or concentrations can be determined by the use of the CGM sensor 112. The CGM sensor 112 utilizes amperometric electrochemical sensor technology to measure analyte levels with three electrodes operably connected to the sensor electronics and are covered by a sensing membrane and a biointerface membrane, which are attached by a clip.

The top ends of the electrodes are in contact with an electrolyte phase (not shown), which may include a free-flowing fluid phase disposed between the sensing membrane and the electrodes. The sensing membrane may include an enzyme, e.g., analyte oxidase, which covers the electrolyte phase. In this exemplary sensor, the counter electrode is provided to balance the current generated by the species being measured at the working electrode. In the case of an analyte oxidase based glucose sensor, the species being measured at the working electrode is H₂O₂. The current that is produced at the working electrode (and flows through the circuitry to the counter electrode) is proportional to the diffusional flux of H₂O₂. Accordingly, a raw signal may be produced that is representative of the concentration of blood glucose in the user's body, and therefore may be utilized to estimate a meaningful blood glucose value. Details of the sensor and associated components are shown and described in U.S. Pat. No. 7,276,029, which is incorporated by reference herein as if fully set forth herein this application. In one embodiment, a continuous analyte sensor from the Dexcom Seven System (manufactured by Dexcom Inc.) can also be utilized with the exemplary embodiments described herein.

In one embodiment of the invention, the following components can be utilized as a diabetes management system: microprocessor enabled devices such as a home computers or a portable handheld computers (e.g., iPhone, iPad, or Android based devices) specifically programmed to receive data from multiple sources (e.g., exercise machine or other sensors) including at least an episodic glucose sensor with test strips such as the Verio blood glucose meter manufactured by LifeScan Inc. or DexCom® SEVEN PLUS® CGM by DexCom Corporation. The microprocessor-enabled device is specifically programmed so that such microprocessor-enabled device is converted into a purpose built diabetes management computer when placed in such mode of operation.

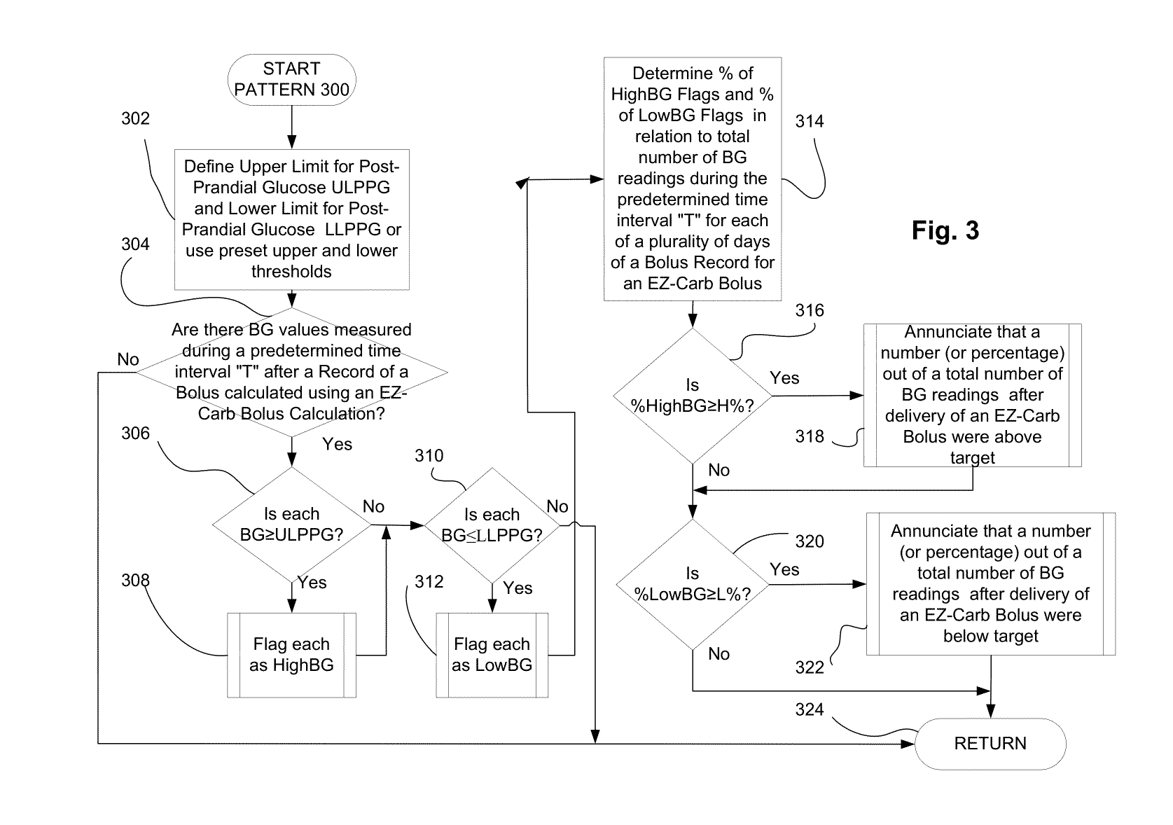

In the system of FIG. 2, the system includes a controller in communication with at least the insulin infusion pump and the at least one glucose monitor, and configured to receive or transmit data regarding glucose levels and dosing of insulin from the at least one glucose monitor and pump for analysis by the controller so that at least one of a plurality of patterns in glucose due to at least one of a plurality of pump commands is determined via the controller. With reference to FIG. 3, applicants note that for the logic processes illustrated herein, it is assumed that the user may set up an upper limit “ULPPG” for post-prandial glucose value and a lower limit “LLPPG” for post-prandial glucose value in step 302. Alternatively, where no upper and lower limits have been set, a default upper limit of about 300 mg/dL and a default lower limit of about 60 mg/dL can be utilized instead.

Returning back to FIG. 3, the controller of the system of FIG. 2 is programmed with the logic illustrated in FIG. 3 to determine (in step 304) whether there is at least one glucose measurement made within a predetermined time interval “T” after occurrence of at least one of a plurality of pump commands. In particular, the system is programmed to find a record of a pump command during a time period of interest such as, for example, during a seven-day period. For each record of pump commands during this time interval of interest, the system looks for a glucose measurement made at about a predetermined time interval “T” (e.g., an interval of about 90 minutes to 240 minutes) after the pump command(s). In the case of FIG. 3, the pump command involves a command for the pump to deliver a bolus based on an automatic calculation made by the pump of (a) the carbohydrates ingested, with (b) the insulin to carb (I:C) ratio, (c) insulin sensitivity factor (ISF), (d) target BG and (e) insulin on board (IOB) that had previously been entered for the current time of day (hereafter referred to as “EZ-Carb Bolus” as described in the Animas User Guide, which is attached in the Appendix).

If the result at step 306 indicates that there is at least one such glucose value (“BG”), which can be from an episodic glucose monitor (“SMBG”) or a continuous glucose monitor (“CGM”) then at step 308, the system flags the at least one glucose measurement as a flagged high measurement whenever the at least one glucose measurement is equal to or greater than a high threshold ULPPG from step 306; alternatively, the system flag at step 312 that the at least one glucose measurement as a flagged low measurement whenever the at least one glucose measurement is equal to or less than a low threshold LLPPG from step 310. At step 314, the logic calculates, if any at all, a percentage of flagged high glucose measurements from total glucose measurements made during the predetermined time interval “T” over a plurality of days in a desired reporting period. Likewise, the logic, at step 314 also calculates a percentage, if any at all, of flagged low glucose measurement from total glucose measurements made during the predetermined time interval “T” over a plurality of days, also in step 314. The logic checks to determine if the percentage of flagged “HighBG” is greater than a first percentage threshold H % at step 316 and if true, annunciate at least a first message in step 318 that a high glucose pattern has been detected in relation to the one of a plurality of pump commands (which for FIG. 3 is for an EZ-Carb Bolus command). The percentage of flagged high glucose measurements can be determined by dividing the number of flagged high glucose measurements by a total number of glucose measurements made during the predetermined time interval “T” for a plurality of days (during the reporting period) multiplied by 100. Conversely, the percentage of flagged low glucose measurements can be obtained by dividing the number of flagged low glucose measurements by a total number of glucose measurements made during the predetermined time interval “T” for a plurality of days (during the reporting period)multiplied by 100. The logic also checks at step 320 to determine if a percentage of flagged “LowBG” is greater than L % or second percentage threshold and if true, a second message in step 322 is annunciated to indicate that a low glucose pattern has been detected in relation to the one of a plurality of pump commands. In this case the pump delivery command is a command for delivery of a bolus (also known as an “EZ-Carb Bolus”) based on calculated carbohydrates (insulin:carbohydrate ratio), preset insulin sensitivity factor, target BG and IOB based on time of the day. Messages that can be annunciated for the impact of this pump command may include, for example: “X out of Y (or alternatively Z % of) glucose readings were below target 1.5-4 hours after delivering an EZ-Carb Bolus” or “X out of Y (or alternatively, Z % of) glucose readings were above target 1.5-4 hours after delivering an EZ-Carb Bolus.”

By virtue of this pattern detection logic 300 in FIG. 3, a user is able to determine the glycemic impact of the utilization of the bolus command for an EZ-Carb Bolus. For example, where the logic 300 is able to detect that certain EZ-Carb Bolus command at a certain time interval during a day causes hyperglycemia or hypoglycemia, the user would be informed so that corrective action(s) towards normoglycemia can be undertaken with respect to this specific pump command.

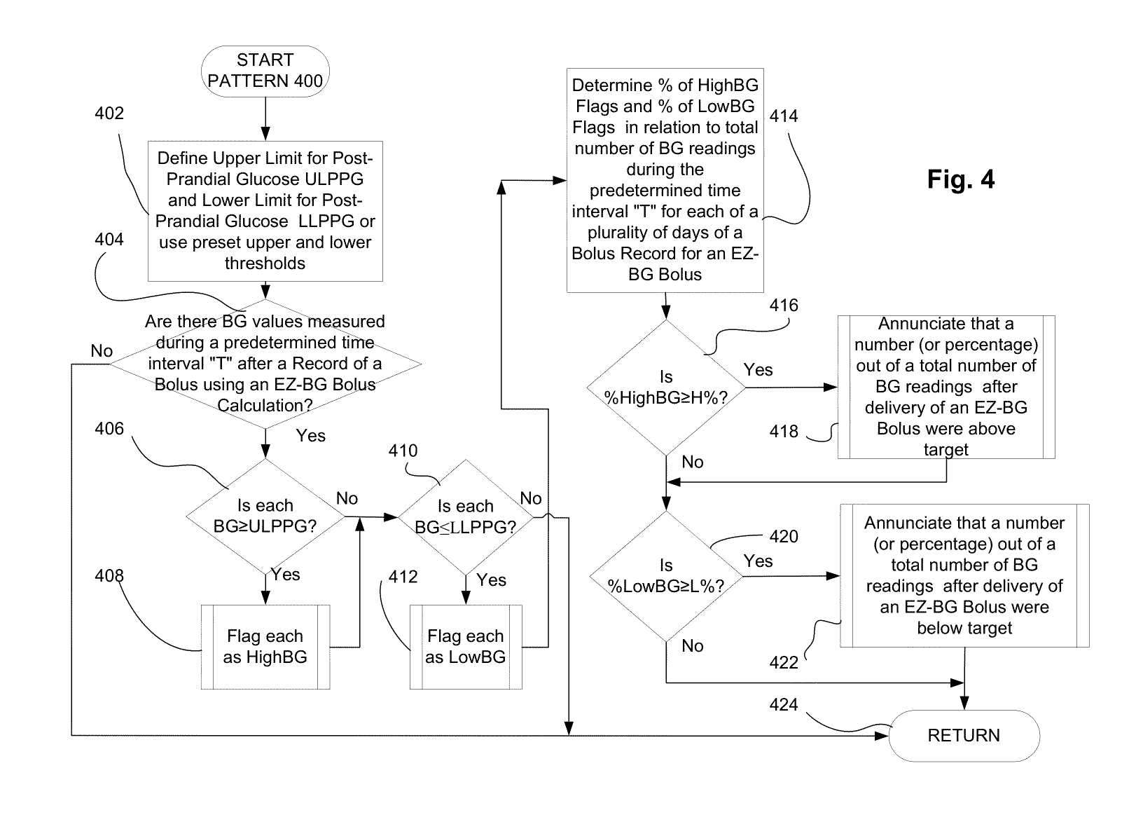

Pattern detection logic similar to pattern detection 300 are also used for patterns 400, 500, 600, 700, 800, 900 (in FIGS. 4-10) but for different pump commands such as, for example, glucose target bolus command, normal bolus command; bolus override command; cannula fill command; pump prime command; or a temporary basal rate command. In particular, certain steps in logical techniques 400, 500, 600, 700, 800, 900, and 1000 are generally identical to pattern 300. For example, steps 402, 502, 602, 702, 802, 902, 1002 are similar to previously described step 302 of FIG. 3; steps 406, 506, 606, 706, 806, 906 are similar to previously described step 306 of FIG. 3; steps 410, 510, 610, 710, 910, 1010 are similar to step 310 of FIG. 3; steps 408, 508, 608, 708, 808, 908 are similar to step 308 of FIG. 3; steps 412, 512, 612, 712, 912, 1012 are similar to previously described step 312 of FIG. 3; steps 416, 516, 616, 716, 816, 916 are similar to previously described step 316 of FIG. 3; steps 420, 520, 620, 720, 920, 1020 are similar to previously described step 320 of FIG. 3. As many of the steps in FIGS. 4-10 are similar, applicants, for the sake of brevity, will now discuss only the steps in FIGS. 4-10 that are dissimilar to the above referenced steps in FIG. 3.

Referring to FIG. 4, step 404 involves the logic determining whether there is at least one glucose value or BG during a predetermined time interval “T” (e.g., from about 90 minutes to 240 minutes) after a pump command to deliver a bolus calculated based on (a) a target blood glucose range for the current time of the day, (b) an insulin sensitivity factor pre-programmed for the current time of the day and (c) IOB. For ease of nomenclature, this bolus command is referred to as an “ezBG Bolus” command in step 404. It is noted that the ezBG Bolus command is generally the same command provided in the Animas User Guide, which is attached in the Appendix. As steps 406-412 are similar to steps 306-312, discussion will not be made with respect to these steps 406-412 but to the remaining steps. Consequently, the logic, at step 414 calculates a percentage, if any at all, of flagged low glucose measurement from total glucose measurements made during the predetermined time interval “T” over a plurality of days. The logic checks to determine if the percentage of flagged “HighBG” is greater than a first percentage threshold H % at step 416 and if true, annunciate at least a first message in step 418 that a high glucose pattern has been detected in relation to the one of a plurality of pump commands (which for FIG. 4 is for an ezBG Bolus command). In pattern detection 400, the first percentage or second percentage threshold can be any percentage less than 100% but the first percentage is preferably about 50% whereas the second percentage is preferably about 5%.

As in the pattern 300, the pattern detection logic 400 may annunciate to the user or

HCP of at least a first message that a high glucose pattern has been detected in relation to the one of a plurality of pump commands whenever the percentage of flagged high glucose measurements is equal to or greater than a first percentage. Alternatively, a second message can be provided to the effect that a low glucose pattern has been detected in relation to the one of a plurality of pump commands whenever the percentage of flagged low glucose measurements is equal to or greater than a second percentage. Messages that can be annunciated for the impact of the ezBG Bolus command may include, for example: “X out of Y (or alternatively, Z % of) glucose readings were below target 1.5-4 hours after delivering an ezBG Bolus” or “X out of Y (or alternatively, Z % of) glucose readings were above target 1.5-4 hours after delivering an ezBG Bolus.” As used here, the term “annunciated” and variations on the root term indicate that an announcement may be provided via text, audio, visual or a combination of all modes of communication to a user, a caretaker of the user or a healthcare provider.

By virtue of this pattern detection 400, a user is able to determine the glycemic impact of the utilization of the bolus command for an ezBG Bolus. For example, where the logic 400 is able to detect that a certain ezBG Bolus command at a certain time interval during a day causes hyperglycemia or hypoglycemia, the user would be informed so that corrective action(s) towards normoglycemia can be undertaken with respect to this specific pump command.

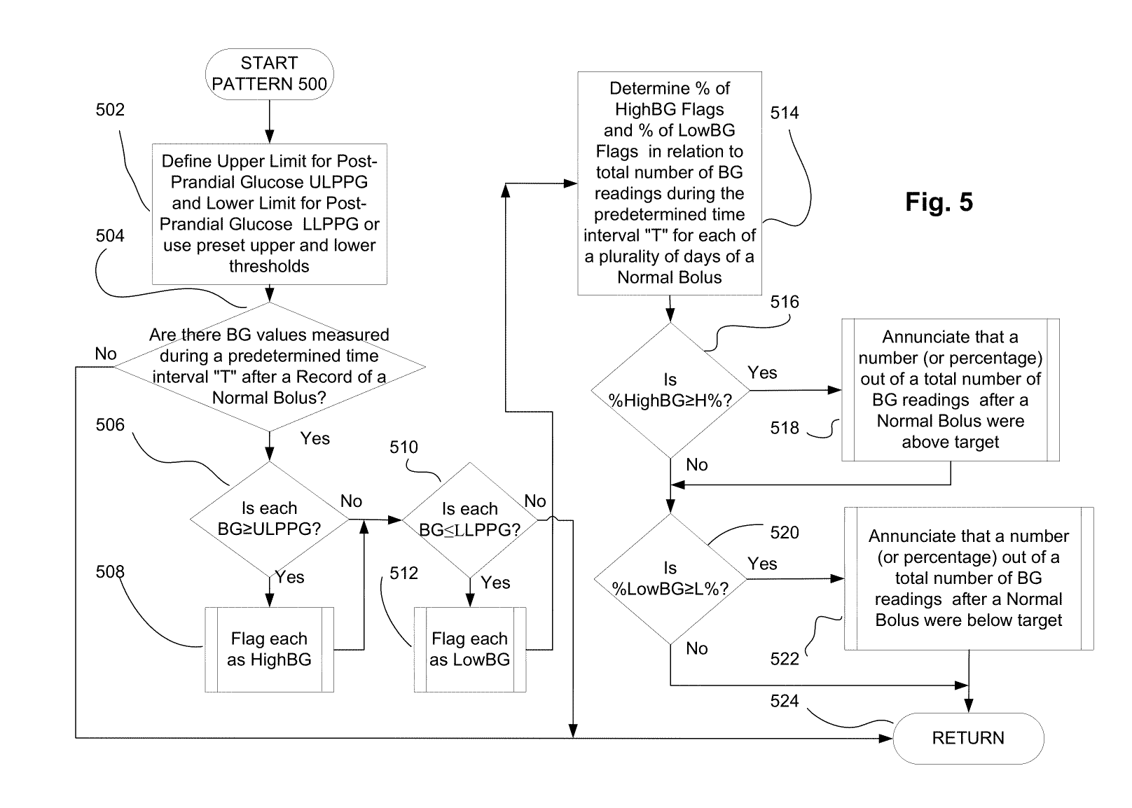

Referring to FIG. 5 to pattern detection logic 500, step 502 is not discussed given that such step similar to step 302 has already been described. Here, step 504 involves the logic determining whether there is at least one glucose value or BG during a predetermined time interval “T” (e.g., from about 90 minutes to 240 minutes) after a pump command to deliver a normal bolus as described in the Animas User Guide, which is attached in the Appendix. As steps 506-512 are similar to steps 306-312, discussion will not be made with respect to these steps 506-512 but to the remaining steps. Consequently, the logic, at step 514 calculates a percentage, if any at all, of flagged low glucose measurement from total glucose measurements made during the predetermined time interval “T” over a plurality of days. The logic checks to determine if the percentage of flagged “HighBG” is greater than a first percentage threshold H % at step 516 and if true, annunciate at least a first message in step 518 that a high glucose pattern has been detected in relation to the one of a plurality of pump commands (which for FIG. 5 is for a normal bolus command). Alternatively, the logic checks to determine if the percentage of flagged low “LowBG” is greater than a second percentage threshold L % in Step 520. If true in step 520, the system annunciates in step 522 at least a message that a low glucose pattern has been detected in relation to the same command. For example, messages to annunciate the impact of certain normal bolus pump commands may include, for example: “X out of Y (or alternatively, Z % of) glucose readings were below target 1.5-4 hours after delivering a normal bolus” or “X out of Y (or alternatively, Z % of) glucose readings were above target 1.5-4 hours after delivering a normal bolus.”

In this pattern detection logic 500, the first percentage or second percentage threshold can be any percentage less than 100% but the first percentage is preferably about 50% whereas the second percentage is preferably about 5%. By virtue of this pattern detection 500, a user is able to determine the glycemic impact of the utilization of the bolus command for a normal Bolus. For example, where the logic 500 is able to detect that a certain normal Bolus command at a certain time interval during a day causes hyperglycemia or hypoglycemia, the user would be informed so that corrective action(s) towards normoglycemia can be undertaken with respect to this specific pump command.

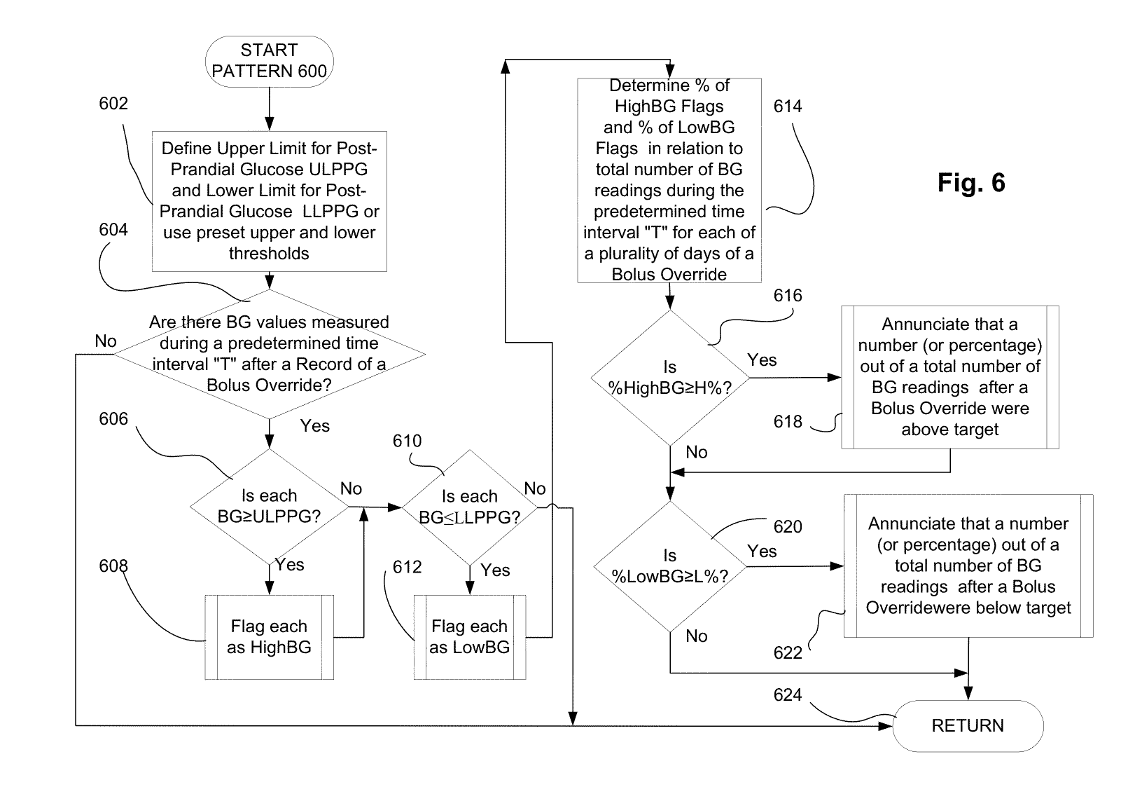

Referring to FIG. 6 to pattern detection logic 600, step 602 is not discussed given that such step similar to step 302 has already been described. Here, step 604 involves the logic determining whether there is at least one glucose value or BG during a predetermined time interval “T” (e.g., from about 90 minutes to 240 minutes) after a command to override a suggested bolus made by the pump. As steps 606-612 are similar to steps 306-312, discussion will not be made with respect to these steps 606-612 but to the remaining steps. Consequently, the logic, at step 614 calculates a percentage, if any at all, of flagged low glucose measurement from total glucose measurements made during the predetermined time interval “T” over a plurality of days. The logic checks to determine if the percentage of flagged “HighBG” is greater than a first percentage threshold H % at step 616 and if true, annunciate at least a first message in step 618 that a high glucose pattern has been detected in relation to the one of a plurality of pump commands (which for FIG. 6 is for a bolus override command). Alternatively, the logic checks to determine if the percentage of flagged low “LowBG” is greater than a second percentage threshold L % in Step 620. If true in step 620, the system annunciates in step 622 at least a message that a low glucose pattern has been detected in relation to the same command. Messages that can be provided to the user or HCPs regarding the impact of certain bolus override commands may include, for example: “X out of Y (or alternatively, Z % of) glucose readings were below target 1.5-4 hours after delivering an insulin bolus inconsistent with the amount suggested by the bolus calculator” or “X out of Y (or alternatively, Z % of) glucose readings were above target 1.5-4 hours after delivering an insulin bolus inconsistent with the amount suggested by the bolus calculator.”

In pattern detection logic 600, the first percentage or second percentage threshold can be any percentage less than 100% but the first percentage is preferably about 50% whereas the second percentage is preferably about 5%. By virtue of this pattern detection 600, a user is able to determine the glycemic impact of the utilization of a bolus override command. For example, where the logic 600 is able to detect that a certain bolus override command at a certain time interval during a day causes hyperglycemia or hypoglycemia, the user would be informed so that corrective action(s) towards normoglycemia can be undertaken with respect to this specific pump command.

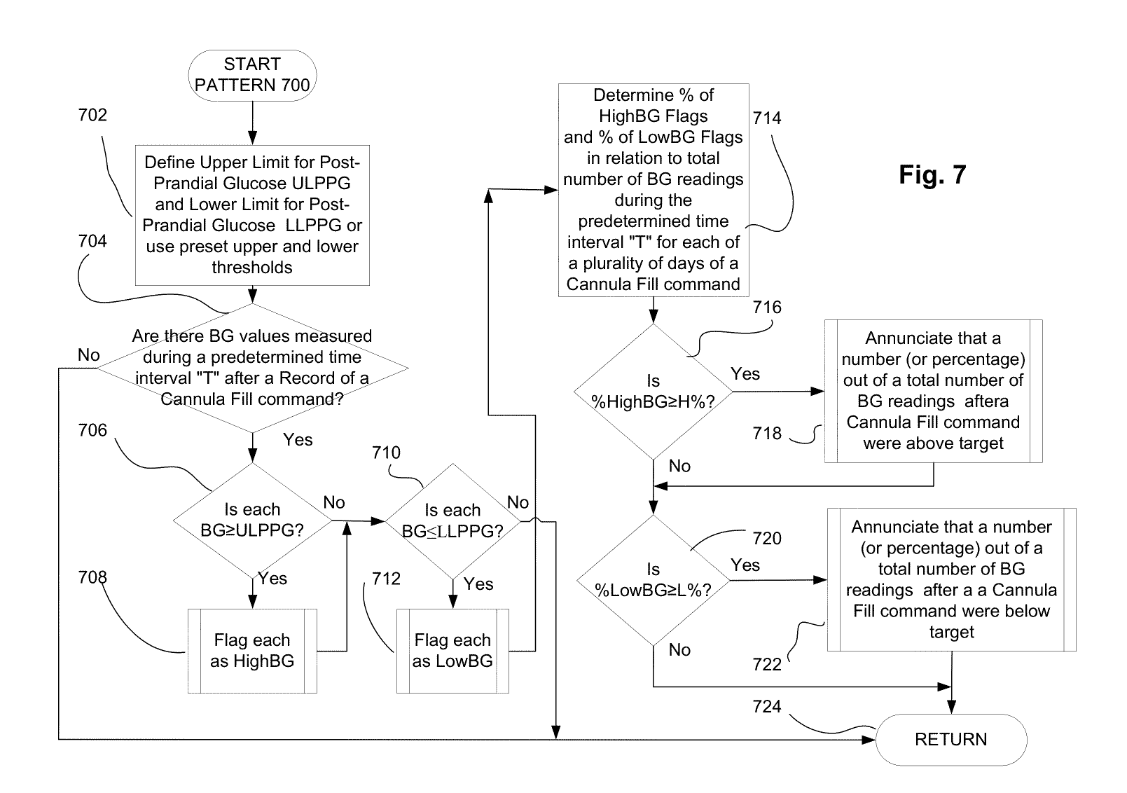

Referring to FIG. 7 to pattern detection logic 700, step 702 is not discussed given that such step similar to step 302 has already been described. Here, step 704 involves the logic determining whether there is at least one glucose value or BG during a predetermined time interval “T” (e.g., from about 90 minutes to 240 minutes) after a pump command to fill a cannula of the inserter set 106 (FIG. 2). As steps 706-712 are similar to steps 306-312, discussion will not be made with respect to these steps 706-712 but to the remaining steps. Consequently, the logic, at step 714 calculates a percentage, if any at all, of flagged low glucose measurement from total glucose measurements made during the predetermined time interval “T” over a plurality of days. The logic checks to determine if the percentage of flagged “HighBG” is greater than a first percentage threshold H % at step 716 and if true, annunciate at least a first message in step 718 that a high glucose pattern has been detected in relation to the one of a plurality of pump commands (which for FIG. 7 is for a cannula fill command). Alternatively, the logic checks to determine if the percentage of flagged low “LowBG” is greater than a second percentage threshold L % in Step 720. If true in step 720, the system annunciates in step 722 at least a message that a low glucose pattern has been detected in relation to the same command. A message that can be provided to the user or HCPs on the impact of certain cannula fill commands may include, for example: “X out of Y (or alternatively, Z % of) glucose readings were above target 1.5-4 hours after filling the cannula.”

In pattern detection logic 700, the first percentage or second percentage threshold can be any percentage less than 100% but the first percentage is preferably about 50% whereas the second percentage is preferably about 5%. By virtue of this pattern detection 400, a user is able to determine the glycemic impact of the utilization of the bolus command for a cannula fill. For example, where the logic 700 is able to detect that a certain cannula fill command at a certain time interval during a day causes hyperglycemia or hypoglycemia, the user would be informed so that corrective action(s) towards normoglycemia can be undertaken with respect to this specific pump command.

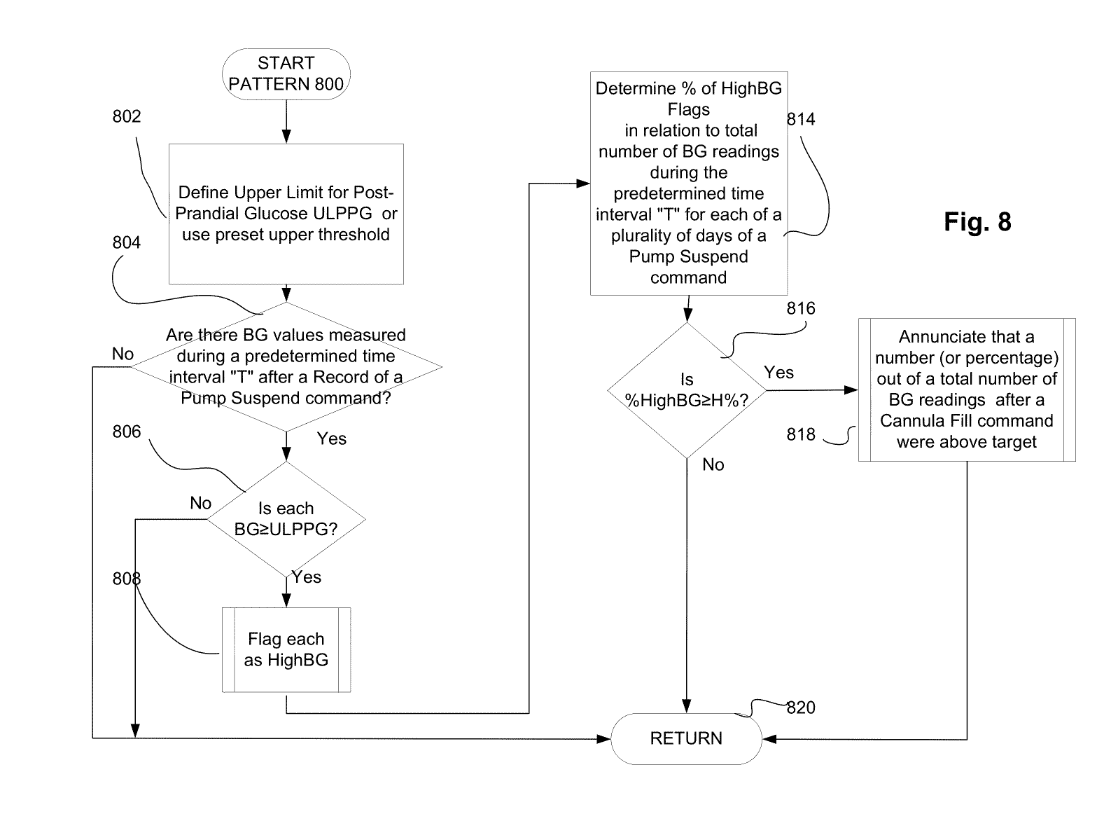

Referring to FIG. 8 to pattern detection logic 800, step 802 is not discussed given that such step similar to step 302 has already been described. Here, step 804 involves the logic determining whether there is at least one glucose value or BG during a predetermined time interval “T” (e.g., from about 90 minutes to 240 minutes) after a command to suspend the delivery of insulin by the pump (“pump suspend command”). As steps 806-812 are similar to steps 306-312, discussion will not be made with respect to these steps 806-812 but to the remaining steps. Consequently, the logic, at step 814 calculates a percentage, if any at all, of flagged high glucose measurement from total glucose measurements made during the predetermined time interval “T” over a plurality of days. The logic checks to determine if the percentage of flagged “HighBG” is greater than a first percentage threshold H % at step 816 and if true, annunciate at least a first message in step 818 that a high glucose pattern has been detected in relation to the one of a plurality of pump commands (which for FIG. 8 is for a pump suspend command). A message that can be annunciated to the user or HCP on the impact of certain pump suspend commands may include, for example: “X out of Y (or alternatively, Z % of) glucose readings were above target after suspending insulin delivery.”

By virtue of this pattern detection 800, a user is able to determine the glycemic impact of the utilization of the pump suspend command. For example, where the logic 800 is able to detect that a pump suspend command at a certain time interval during a day causes hyperglycemia (due to insufficient insulin to control blood glucose), the user would be informed so that corrective action(s) towards normoglycemia can be undertaken with respect to this specific pump command.

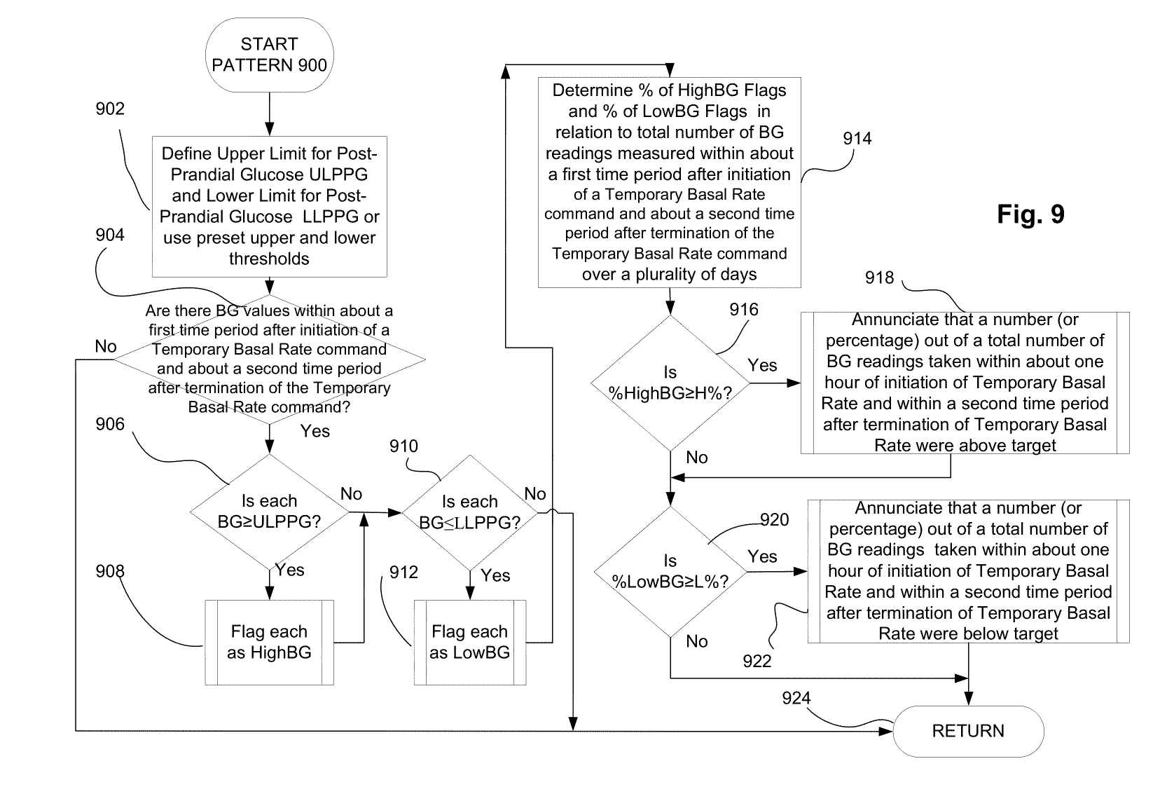

Referring to FIG. 9 to pattern detection logic 900, step 902 is not discussed given that such step similar to step 302 has already been described. Here, step 904 involves the logic determining whether there is at least one glucose value or BG during a first time interval “T1” after initiation of a temporary basal command and a second time interval “T2” after termination of the basal rate command. This feature allows the user to increase the user's active basal delivery rate for events such as sick days or decrease for events such as exercise. As steps 906-912 are similar to steps 306-312, discussion will not be made with respect to these steps 906-912 but to the remaining steps. Consequently, the logic, at step 914 calculates a percentage, if any at all, of flagged low glucose measurement from total glucose measurements made during the predetermined time interval “T” over a plurality of days. The logic checks to determine if the percentage of flagged “HighBG” is greater than a first percentage threshold H % at step 916 and if true, annunciates, at least a first message in step 918 that a high glucose pattern has been detected in relation to the temporary basal rate command. Alternatively, the logic checks to determine if the percentage of flagged low “LowBG” is greater than a second percentage threshold L % in Step 920. If true in step 920, the system annunciates at least a message that a low glucose pattern has been detected in relation to the temporary basal command. Messages that can be annunciated to the user or HCPs regarding the impact of certain pump suspend commands may include, for example: “X out of Y (or alternatively, Z % of) glucose readings results were below target after setting a temporary basal rate” or “X out of Y (or alternatively, Z % of) glucose readings were above target after setting a temporary basal rate.”

In pattern detection 900, the first percentage or second percentage threshold can be any percentage less than 100% but the first percentage is preferably about 50% whereas the second percentage is preferably about 5%. By virtue of this pattern detection 900, a user is able to determine the glycemic impact of the utilization of the basal rate command. For example, where the logic 900 is able to detect that a certain basal rate command at a certain time interval during a day causes hyperglycemia or hypoglycemia, the user would be informed so that corrective action(s) towards normoglycemia can be undertaken with respect to this specific pump command.

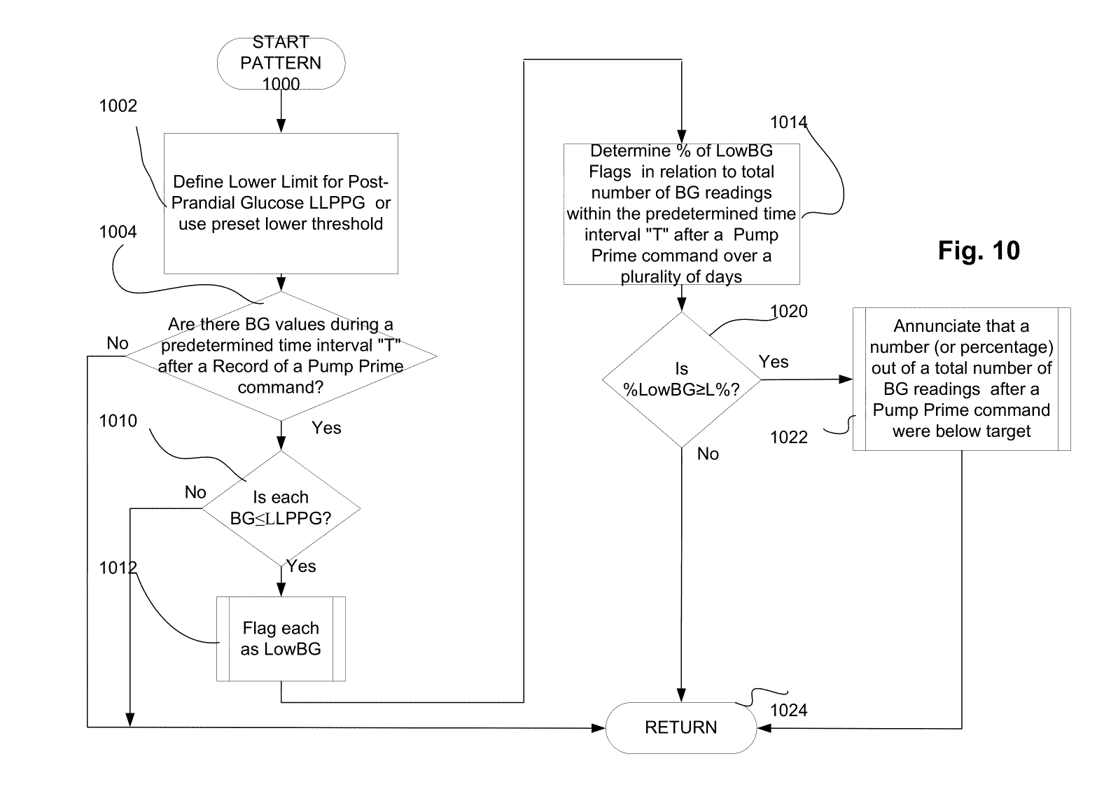

Referring to FIG. 10 to pattern detection logic 1000, step 1002 is not discussed given that such step similar to step 302 has already been described. Here, step 1004 involves the logic determining whether there is at least one glucose value or BG during a predetermined time interval “T” (e.g., from about 90 minutes to 240 minutes) after a command to prime the pump (“pump prime command”). As steps 1010-1012 are similar to steps 310-312, discussion will not be made with respect to these steps 1010-1012 but to the remaining steps. Consequently, the logic, at step 1014 calculates a percentage, if any at all, of flagged low glucose measurement from total glucose measurements made during the predetermined time interval “T” over a plurality of days. The logic checks to determine if the percentage of flagged “LowBG” is greater than a second percentage threshold L % at step 1020 and if true, annunciate at least a first message in step 1022 that a low glucose pattern has been detected in relation to the pump prime command. By virtue of this pattern detection 1000, a user is able to determine the glycemic impact of the utilization of pump prime command for hypoglycemia. For example, where the logic 1000 is able to detect that a certain prime command at a certain time interval during a day causes hypoglycemia, the user would be informed so that corrective action towards normoglycemia can be undertaken with respect to this specific pump command. A message that can be annunciated to the user or HCPs may include, for example: “X out of Y (or alternatively, Z % of) glucose readings were below target within 2 hours after priming the insulin pump.”

It is noted that recommendations, warnings and compliance updates may be annunciated to a user in a suitable medium, such as a visual medium in the form of a display screen, printed paper, or in the form of an audio message to the user or subject. In one embodiment, as shown in FIG. 6, a display screen can be utilized to annunciate to the subject or user the hypoglycemic states of the subject during a reporting period. As used herein, the term “user” is intended to indicate primarily a mammalian subject (e.g., a person) who has diabetes but which term may also include a caretaker or a healthcare provider who is operating the glucose monitor or the insulin pump on behalf of the diabetes subject.

It is noted that the various methods described herein can be used to generate software codes using off-the-shelf software development tools such as, for example, Visual Studio 6.0, Windows 2000 Server, and SQL Server 2000. The methods, however, may be transformed into other software languages depending on the requirements and the availability of new software languages for coding the methods. Additionally, the various methods described, once transformed into suitable software codes, may be embodied in any computer-readable storage medium that, when executed by a suitable microprocessor or computer, are operable to carry out the steps described in these methods along with any other necessary steps.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

What is claimed is:

1. A system for management of diabetes of a subject, the system comprising:
at least one glucose monitor for measurements of the glucose levels of the subject;
an insulin infusion pump configured for communication with the at least one glucose monitor and delivery of insulin to the subject; and
a controller in communication with at least the insulin infusion pump and the at least one glucose monitor, the controller being configured to receive or transmit data regarding glucose levels and dosing of insulin from the at least one glucose monitor and pump for analysis by the controller so that at least one of a plurality of patterns in glucose due to at least one of a plurality of pump commands is determined via the controller by:
determination of whether there is at least one glucose measurement made within a predetermined time interval after occurrence of one of a plurality of pump commands;
flag the at least one glucose measurement as a flagged high measurement whenever the at least one glucose measurement is equal to or greater than a high threshold;
flag the at least one glucose measurement as a flagged low measurement whenever the at least one glucose measurement is equal to or less than a low threshold;
calculate a percentage of flagged high glucose measurements from total glucose measurements made during the predetermined time interval over a plurality of days;
calculate a percentage of flagged low glucose measurement from total glucose measurements made during the predetermined time interval over a plurality of days;
annunciate at least a first message that a high glucose pattern has been detected in relation to the one of a plurality of pump commands whenever the percentage of flagged high glucose measurements is equal to or greater than a first percentage or a second message that a low glucose pattern has been detected in relation to the one of a plurality of pump commands whenever the percentage of flagged low glucose measurements is equal to or greater than a second percentage.

2. The system of claim 1, in which the plurality of pump commands comprises a command for:
a bolus based on a carbohydrate calculator, a bolus based on measured glucose values, an override of a programmed bolus, a programmed bolus, or a temporary basal rate.

3. The system of claim 1, in which the first percentage comprises about 50% and the second percentage comprises about 5%.

4. A method for managing diabetes of a subject with at least a glucose monitor and infusion pump, the method comprising:
conducting, with the glucose monitor, a plurality of glucose measurements of the subject;
storing the plurality of glucose measurements in a memory;
determining whether there is at least one glucose measurement made within a predetermined time interval after occurrence of one of a plurality of pump commands;
flagging the at least one glucose measurement as a flagged high measurement whenever the at least one glucose measurement is equal to or greater than a high threshold;
flagging the at least one glucose measurement as a flagged low measurement whenever the at least one glucose measurement is equal to or less than a low threshold;
calculating a percentage of flagged high glucose measurements from total glucose measurements made during the predetermined time interval over a plurality of days;
calculating a percentage of flagged low glucose measurement from total glucose measurements made during the predetermined time interval over a plurality of days;
annunciating at least a first message that a high glucose pattern has been detected in relation to the one of a plurality of pump commands whenever the percentage of flagged high glucose measurements is equal to or greater than a first percentage or a second message that a low glucose pattern has been detected in relation to the one of a plurality of pump commands whenever the percentage of flagged low glucose measurements is equal to or greater than a second percentage.

5. The method of claim 4, in which the calculating of the percentage of flagged high glucose measurements comprises dividing the number of flagged high glucose measurements by a total number of glucose measurements made during the predetermined time interval for a plurality of days multiplied by 100 and the calculating of the percentage of flagged low glucose measurements comprises dividing the number of flagged low glucose measurements by a total number of glucose measurements made during the predetermined time interval for a plurality of days multiplied by 100.

6. The method of claim 4, in which the plurality of pump commands comprises a command for:
a bolus based on a carbohydrate calculator, a bolus based on measured glucose values, an override of a programmed bolus, a programmed bolus, or a temporary basal rate.

7. The method of claim 4, in which the first percentage comprises about 50% and the second percentage comprises about 5%.

8. A method for managing diabetes of a subject with at least a glucose monitor and infusion pump, the method comprising:
conducting, with the glucose monitor, a plurality of glucose measurements of the subject;
storing the plurality of glucose measurements in a memory;
determining whether there is at least one glucose measurement made during a predetermined time period after occurrence of a pump suspend command and a second predetermined time period after occurrence of a pump resume command;
flagging the at least one glucose measurement as a flagged high measurement whenever the at least one glucose measurement is equal to or greater than a high threshold;
calculating a percentage of flagged high glucose measurements from total glucose measurements made during the first and second predetermined time periods over a plurality of days; and
annunciating a first message that a high glucose pattern has been detected in relation to the pump suspend command whenever the percentage of flagged high glucose measurements is equal to or greater than a first percentage.

9. The method of claim 8, in which the first and second time period comprise equal time intervals.

10. The method of claim 8, in which the first and second time period comprise unequal time intervals.

11. The method of claim 8, in which each of the first and second time periods comprises about one hour in duration.

12. The method of claim 8, in which the calculating of the percentage of flagged high glucose measurements comprises dividing the number of flagged high glucose measurements by a total number of glucose measurements made during the predetermined time periods for a plurality of days multiplied by 100.

13. The method of claim 8, in which the first percentage comprises about 50%.

14. A method for managing diabetes of a subject with at least a glucose monitor and infusion pump, the method comprising:
conducting, with the glucose monitor, a plurality of glucose measurements of the subject;
storing the plurality of glucose measurements in a memory;
determining whether there is at least one glucose measurement made within a predetermined time interval after occurrence of a pump prime command;
flagging the at least one glucose measurement as a flagged low measurement whenever the at least one glucose measurement is equal to or less than a low threshold;
calculating a percentage of flagged low glucose measurement from total glucose measurements made during the predetermined time interval over a plurality of days; and
annunciating a second message that a low glucose pattern has been detected in relation to the pump prime command whenever the percentage of flagged low glucose measurements is equal to or greater than a second percentage.

15. The method of claim 14, in which the calculating of the percentage of flagged low glucose measurements comprises dividing the number of flagged low glucose measurements by a total number of glucose measurements made during the predetermined time interval for a plurality of days multiplied by 100.

16. The method of claim 14, in which the second percentage comprises about 5%

17. The method of claim 14, in which the predetermined time interval comprises about 2 hours.
